(19) United States
(12) Patent Application Publication
(10) Pub. No.: US 2005/0158777 A1
(43) Pub. Date: Jul. 21, 2005

(54) Method of isolating nucleic acid by using carbon nanotube

(76) Inventors: Yoon-kyoung Cho, Suwon-si (KR); Joon-ho Kim, Seongnam-si (KR); Jung-joo Hwang, Suwon-si (KR); Wan-jun Park, Seoul (KR); Geun-bae Lim, Pohang-si (KR)

Correspondence Address: CANTOR COLBURN, LLP, 55 GRIFFIN ROAD SOUTH, BLOOMFIELD, CT, 06002, US

(21) Appl. No.: 11/024,315

(22) Filed: Dec. 28, 2004

(30) Foreign Application Priority Data
Jan. 2, 2004 (KR) 2004-42

(51) Int. Cl. C12Q 1/68; C07H 21/04

(52) U.S. Cl. 435/6; 536/25.4

(57) ABSTRACT

A method of isolating nucleic acid using a carbon nanotube is provided. The method includes contacting a mixture of a sample containing nucleic acid and a salt solution with a carbon nanotube to form a nucleic acid-carbon nanotube composite; washing the nucleic acid-carbon nanotube composite with a washing buffer; and eluting the nucleic acid from the nucleic acid-carbon nanotube composite.

### BACKGROUND OF THE INVENTION

This application claims the benefit of Korean Patent Application No. 10-2004-0000042, filed on Jan. 2, 2004, in the Korean Intellectual Property Office, the disclosure of which is incorporated herein in its entirety by reference.

1. Field of the Invention

The present invention relates to a method of isolating nucleic acid by using a carbon nanotube.

2. Description of the Related Art

A conventional method of isolating nucleic acid using a solid material is known. For example, U.S. Pat. No. 5,234,809 discloses a method of isolating nucleic acid using a solid material to which the nucleic acid binds. Specifically, the method includes mixing a starting material containing nucleic acid, a chaotropic material, and a solid material to which the nucleic acid binds; isolating the solid material having the nucleic acid bound thereto from the liguid materials; and washing the solid material-nucleic acid composite. Examples of chaotropic materials include guanidinium salt, sodium iodide, sodium thiocyanate, and urea. Example of the solid material includes silica particles.

The above method must use a chaotropic material. In other words, when a chaotropic material is not used, the nucleic acid does not bind to the solid material. Unfortunately, chaotropic materials are harmful to the human body and thus must be handled carefully. Further, a chaotropic material acts as an interference material in a subsequent process such as a PCR and thus must be removed from the isolated nucleic acid during or after isolating the nucleic acid.

U.S. Pat. No. 6,291,166 discloses a method of archiving nucleic acid using a solid matrix. Specifically, the method includes a) irreversibly binding a single or multiple-strand nucleic acid contained in an aqueous specimen to a solid matrix, the solid matrix being a specific binding material in which an electropositive material is hydrophilically modified; b) manipulating the nucleic acid bound to the solid matrix; and c) storing the nucleic acid bound to the solid matrix. Examples of the solid matrix include silicon (Si), boron (B), and aluminum (Al). The process of hydrophilically modifying the electropositive material may be achieved by using a basic solution such as NaOH. In the method, step (b) includes an enzyme reaction, hybridization, signal amplification, and target amplification using the nucleic acid irreversibly bound to the solid matrix. PCR, SDA, NASBA, IsoCR, CRCA, Q beta replicase, or a branched chain DNA method may be used for the target amplification. Since the nucleic acid irreversibly binds to the solid matrix, delayed analysis after storing or repeated analysis of the nucleic acid-solid matrix composite is possible. However, a material having an electropositive surface such as aluminum must be hydrophilically modified using the basic material such as NaOH, and the nucleic acid irreversibly binds to the hydrophilic aluminum, thereby making separation of the nucleic acid from the aluminum impossible.

In addition, U.S. Pat. No. 5,898,071 discloses a method of non-specifically reversibly binding nucleic acid to a magnetic microparticle having a surface coated with a functional group. Specifically, the method includes mixing a magnetic microparticle having a surface coated with a functional group, reversibly binding to a polynucleotide and a solution containing the polynucleotide, and binding the polynucleotide on the magnetic microparticle by adjusting the concentrations of a salt and PEG in the mixture. The magnetic microparticle may be a magnetic particle coated with a carboxylic group. Since the method must use the magnetic particle, when the method is performed in a microchannel, the microchannel can become clogged. Also, when the magnetic particle is settled, mixing is required.

The inventors of the present invention found a method of reversibly binding nucleic acid to a carbon nanotube while studying a method of isolating nucleic acid based on the above-described conventional techniques, and thus completed the present invention.

### SUMMARY OF THE INVENTION

The present invention provides a method of isolating nucleic acid by using a carbon nanotube capable of efficiently isolating the nucleic acid.

According to an aspect of the present invention, there is provided a method of isolating nucleic acid using a carbon nanotube, the method comprising:
contacting a mixture of a sample containing nucleic acid and a salt solution with a carbon nanotube to form a nucleic acid-carbon nanotube composite;
washing the nucleic acid-carbon nanotube composite with a washing buffer; and
eluting the nucleic acid from the nucleic acid-carbon nanotube composite.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates an electrophoresis result of DNA isolated by a method according to an embodiment of the present invention;

FIG. 2A illustrates an electrophoresis result of a PCR product obtained by a PCR using HBV plasmid DNA isolated by a method according to an embodiment of the present invention, as a template;

FIG. 2B is a graph illustrating the concentration of a PCR product obtained by performing a PCR using DNAs isolated from DNA samples having different initial concentrations by a method according to an embodiment of the present invention as templates;

FIG. 3 is an electron microscope photograph showing a carbon nanotube formed on an aluminum substrate;

FIG. 4 schematically illustrates a polymer chamber used in the present invention; and

FIG. 5 illustrates an electrophoresis result of a PCR product obtained by a PCR using HBV plasmid DNA isolated by using an aluminum substrate on which a carbon nanotube is formed as a template.

### DETAILED DESCRIPTION OF THE INVENTION

According to an embodiment of the present invention, there is provided a method of isolating nucleic acid using a carbon nanotube, the method including contacting a mixture of a sample containing nucleic acid and a salt solution with a carbon nanotube to form a nucleic acid-carbon nanotube composite, washing the nucleic acid-carbon nanotube composite with a washing buffer, and eluting the nucleic acid from the nucleic acid-carbon nanotube composite.

The sample containing nucleic acid may be a biological material containing nucleic acid. Examples of the biological sample include whole blood, serum, buffy coat, urine, feces, cerebrospinal fluid, sperm, saliva, tissue, cell culture, and the like. The sample may also be a nonbiological material containing nucleic acid. When using a biological sample, pre-treatment may be performed when it is difficult to directly contact the nucleic acid with the carbon nanotube due to obstacles such as a cell wall, a cell membrane, and an envelope. Such pre-treatment may be, for example, a material destroying a cell such as a detergent and an organic solvent. For example, a cell is disintegrated with NaOH, neutralized, and then isolated with the salt solution, such as NaCl, used in the present invention.

The salt solution used in the present invention may be a solution containing at least one salt selected from NaCl, MgCl₂, KCl, CaCl₂, and a combination thereof. The concentration of the salt may be 0.5-5 M.

A carbon nanotube is a material having a tube shape, in which carbon binds to another carbon to form a hexagonal honeycomb. The carbon nanotube is a very small sized material having a diameter on the scale of nanometers. Since Kroto and Smalley had first found Fullerene (gathered 60 carbon atoms: C60), which is one type of carbon allotropes, Dr. Iijima of an annex research institute of NEC found a carbon nanotube of a slender bamboo tube shape while analysing through a TEM a carbon mass formed on a graphite cathode using arc-discharge. Carbon nanotubes can be mass-produced through arc-discharge, laser vaporization, plasma enhanced chemical vapor deposition, thermal chemical vapor deposition, vapor phase growth, electrolysis, flame synthesis, and the like. Carbon nanotubes have good mechanical properties, good electric selectivity, good electric field emission property, high efficiency hydrogen storing medium property, and the like. Recently, attempts to apply the carbon nanotube as a biosensor using its electrical property are increasing. The present invention relates to a method of isolating nucleic acid by using a carbon nanotube and provides a method in which a surface of a biosensor may be also utilized for separating and isolating nucleic acid when the biosensor using the carbon nanotube is later developed. The carbon nanotube used in the present invention may have a surface coated with a carboxylic group. The carbon nanotube having the surface coated with the carboxylic group may be prepared, for example, by oxidizing isolated single wall carbon nanotubes (SWNT) to generate the carboxylic group on ends and a wall surface of the tubes [McCreery, R. L. In Electroanalytical Chemistry; Bard, A. J., Ed.; Marcel Dekker: New York, 1991; ch. 17, pp 221-374].

In the method according to an embodiment of the present invention, the washing may be performed with a washing buffer containing ethanol and EDTA. The washing buffer may be an aqueous solution containing 70% ethanol and 10 mM EDTA.

In the method according to an embodiment of the present invention, the elution may be performed using at least one selected from water, Tris-buffer, and PBS.

In the mixing, 0-40% of PEG may be further included in the mixture.

The present invention will be described in greater detail with reference to the following examples. The following examples are for illustrative purposes and are not intended to limit the scope of the invention.

### EXAMPLES

### Example 1

### Isolation of Nucleic Acid Using Carbon Nanotube Powder

In the present example, DNA was isolated from a sample containing DNA with a carbon nanotube. Commercially available SWNT particles (Iljinnanotech, Korea) (diameter: 0.8-1.3 nm, length: 2-21 nm) and a carbon nanotube (diameter: 0.8-1.2 nm, length: 2-20 nm) obtained by treating the same with an acid were used as carbon nanotubes. For comparison, magnetic particles coated with a carboxylic group, DynabeadsR (Dynal Biotech Inc.) (diameter: 2.8 μm±0.2, specific surface area: 2-5 m²/g) were used. The DNA used was pBR322 plasmid DNA (about 4.3 kb, Promega). The experimental procedures were as follows.
1. First, the SWNT powder was dissolved in PBS buffer of pH 7.4 (concentration: 38.4 μg/l). Then, 10 μl of the carbon nanotube solution was taken and placed in an ependorff tube.
2. 100 μl of EDTA buffer was added to the carbon nanotube solution, the resulting solution was centrifuged to immerse carbon nanotube particles, and then a supernatant was removed. This process was repeated three times.
3. pBR322 plasmid DNA was dissolved in distilled water to prepare a 10 ng/μl solution.
4. 100 μl of the DNA solution in distilled water solution was mixed with 100 μl of a 2.5 M NaCl solution containing 20% PEG 8000 (Aldrich).
5. The mixture was mixed with the carbon nanotube prepared above in step 2 and left at room temperature for 5 minutes.
6. The solution obtained in step 5 was centrifuged to immerse the carbon nanotube, and then a supernatant was removed.
7.70% ethanol and 10 mM EDTA solution were injected into the tube containing the carbon nanotube and centrifuged to immerse the carbon nanotube, and then a supernatant was removed. This process was repeated three times.
8.50 μl of distilled water was added to the tube and centrifuged to immerse the carbon nanotube, and then a supernatant was collected.
9. The isolated DNA was subjected to electrophoresis on agarose gel, thereby confirming the existence of DNA.

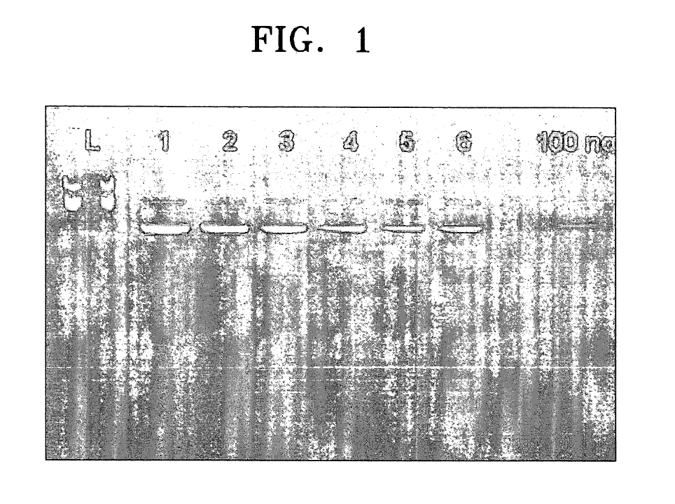

The result is illustrated in FIG. 1. In FIG. 1, lanes 1 and 2 were obtained using Dynabead™, lanes 3 and 4 were obtained using a carbon nanotube, and lanes 5 and 6 were obtained using an acid treated carbon nanotube. FIG. 1 shows that DNA can be isolated using a carbon nanotube.

### Example 2

### Isolation of HBV Plasmid DNA Using a Carbon Nanotube

In the present example, plasmid DNA was isolated using a carbon nanotube, and then PCR was performed using the isolated product as a template.

Commercially available SWNT particles (Iljinnanotech, Korea) (diameter: 0.8-1.3 nm, length: 2-21 nm) and a carbon nanotube (diameter: 0.8-1.2 nm, length: 2-20 nm) obtained by treating the same with an acid were used as carbon nanotubes. The plasmid DNA was HBV plasmid DNA (about 7.3 kb, ATCC No. 45020D). The isolation process was the same as in Example 1.

A PCR was performed using the isolated DNA as a template and oligonucleotides of SEQ ID No. 1 and 2 as primers (length of an amplified product: about 100 bp) on MJ Research PTC-100 apparatus. The PCR was performed at 95° C. for 20 seconds, at 58° C. for 30 seconds, at 72° C. for 40 seconds, and repeated 40 times. After PCR was completed, the PCR product was analysed using Agilent 2100 Bioanalyzer (Agilent) electrophoresis apparatus.

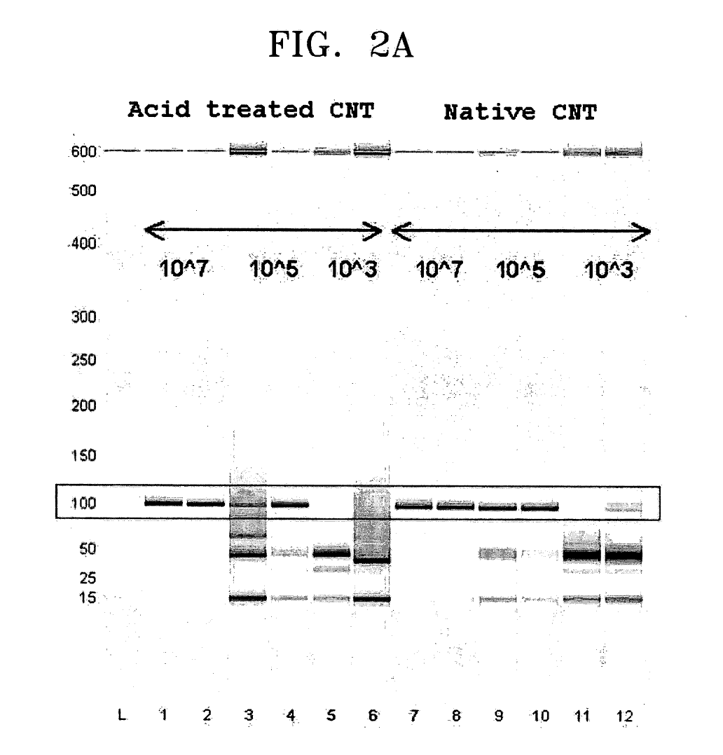

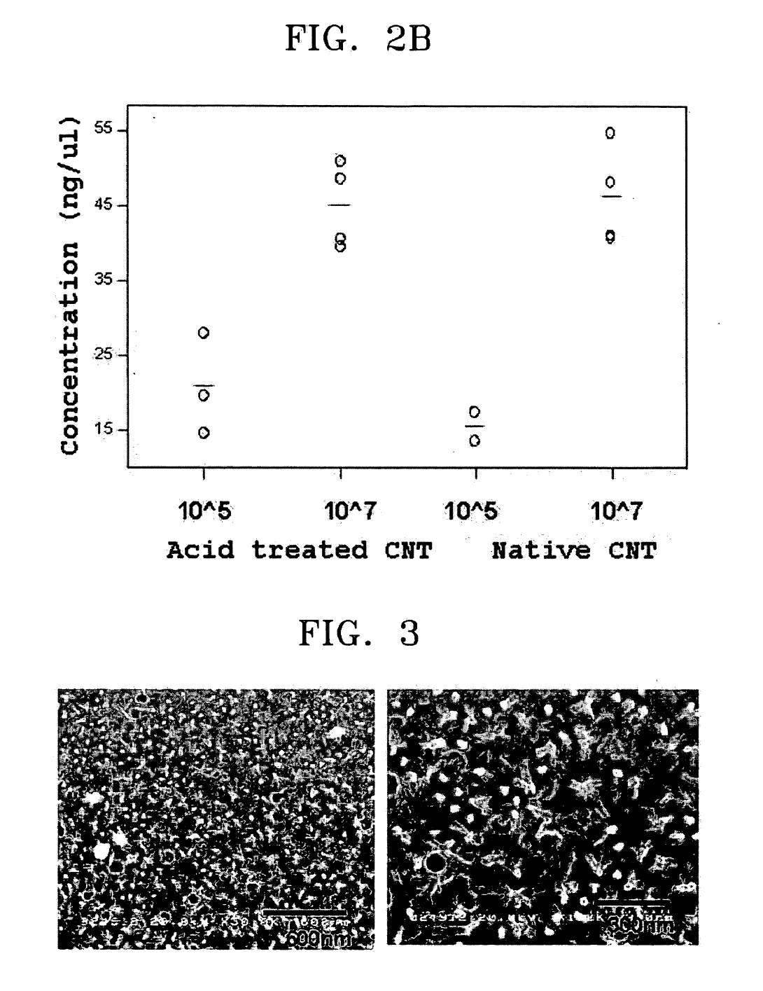

The result is illustrated in FIG. 2A. Referring to FIG. 2A, when using samples having initial HBV plasmid DNA concentrations of 10⁷ copies/μl, 10⁵ copies/μl, and 10³ copies/μl, respectively, in an amount of 100 μl, PCR products could be identified in the samples having an initial concentration of 10⁵ copies/μl or higher. FIG. 2B illustrates the concentrations of the isolated DNA when the initial HBV plasmid DNA concentrations were 10⁷ copies/μl and 10⁵ copies/μl.

### Example 3

### Isolation of HBV Plasmid DNA Using a Carbon Nanotube Formed on a Substrate

1. Formation of a Carbon Nanotube on an Aluminium Substrate

First, a porous oxide film having pores with a depth of 500 nm and an average diameter of 20-30 nm was prepared using an aluminium bulk film (thickness: 0.5 mm). Then, a carbon nanotube was grown on the porous oxide film using a carbon source C₂H₂ in a thermal CVD apparatus at a reaction temperature of 600° C. Thereafter, deposited carbon was removed via physical etching, and about 150 nm of the porous oxide film was exposed through chemical etching (using a wet etching solution). As a result, the aluminium substrate contains carbon nanotubes at a density of 10⁸-10¹⁰ carbon nanotubes/cm² (refer to FIG. 3).

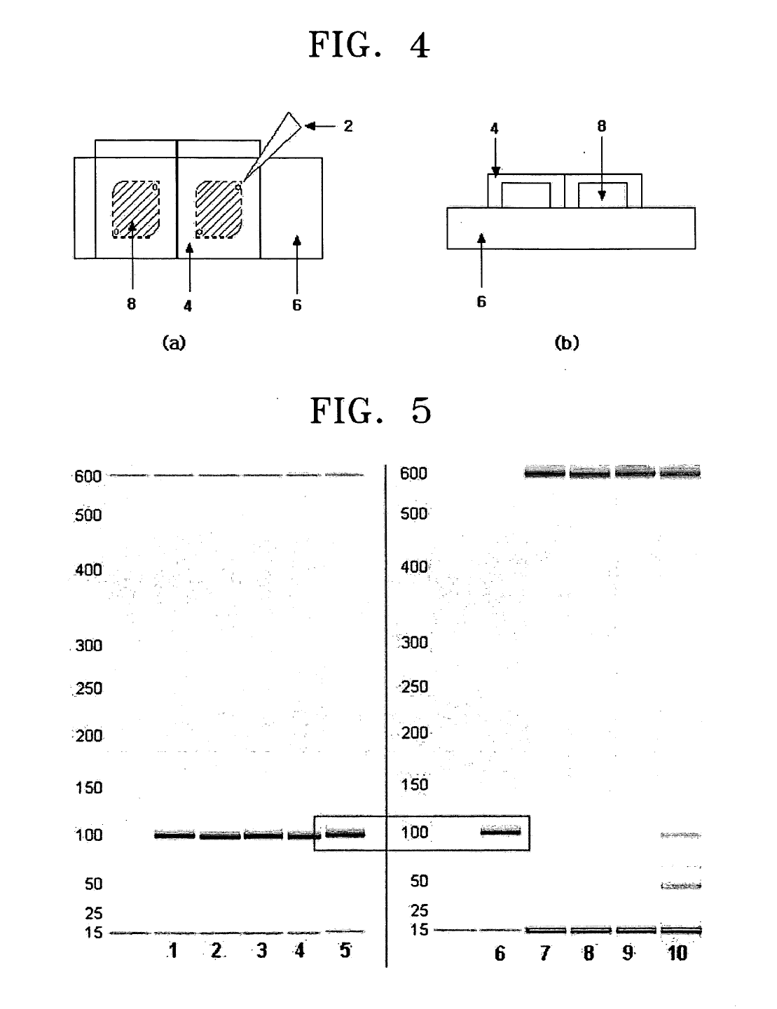

A polymer chamber housing 4 having a space of 1.6 mm×1.6 mm×0.4 mm and having an inlet and an outlet for a fluid was attached to the aluminium substrate 6 on which the carbon nanotube was formed. Thus formed polymerisation chamber 8 was used as a chamber for isolation and polymerisation of DNA (refer to FIG. 4). Referring to FIG. 4, a sample containing DNA was injected into the polymerisation chamber 8 via the sample inlet using a micropippet 2. In FIG. 4, (a) is a plan view of the polymer chamber housing 4 attached to the aluminium substrate 6 on which the carbon nanotube was formed, and (b) is a side sectional view thereof.

2. Isolation of DNA and PCR Using the Isolated DNA as a Template

The aluminium substrate on which the carbon nanotube was formed was used as a solid substrate. The DNA used was HBV plasmid DNA (about 7.3 kb, ATCC No. 45020D). The experimental procedures were as follows.
1. First, HBV plasmid DNA was dissolved in distilled water.
2. 100 μl of the DNA solution in distilled water was mixed with 100 μl of a 2.5 M NaCl solution containing 20% PEG.
3. 180 μl of the mixture was injected into the carbon nanotube included in the polymer chamber. The chamber having an inlet and an outlet for a sample and having a space of 1.6 mm×1.6 mm×0.4 mm was prepared by attaching the polymer chamber housing to the substrate.
4. After injecting the mixture containing DNA, the mixture was left at room temperature for 10 minutes.
5. Then, the sample solution was removed from the polymer chamber.
6. The chamber was washed three times with 70% ethanol and 10 mM EDTA solution.
7. 180 μl of distilled water was injected into the chamber to elute the bound DNA, and the eluted solution was collected.
8. 10 μl of 180 μl of the eluted solution was used as a template to perform a PCR on MJ Research PTC-100 apparatus. The PCR was repeated 40 times at 95° C. for 20 seconds, at 58° C. for 30 seconds, and at 72° C. for 40 seconds.
9. After the PCR was completed, the PCR product was analysed using Agilent 2100 Bioanalyzer (Agilent) electrophoresis apparatus.

FIG. 5 is an electrophoresis result of the PCR product obtained through a PCR using the isolated HBV plasmid DNA as a template. As is apparent from FIG. 5, DNA was very efficiently isolated. In FIG. 5, lanes 1, 2, 3, and 4 are the results of comparison experiments performed in the same manner as in Example 2. Lanes 1 and 2 are the results for natural SWNT, and lanes 3 and 4 are the results for the acid treated carbon nanotube. Lanes 5 and 6 are the results obtained using the carbon nanotube formed on the substrate, performed in the present example. Lanes 7, 8, 9, and 10 are experimental results for negative controls.

Thus, it is demonstrated by the above Examples of the present invention that nucleic acid can be isolated using an aluminium substrate on which a carbon nanotube is formed. Also, DNA isolated by the method according to an embodiment of the present invention can be directly used as a template for a PCR.

The carbon nanotube used in the present invention can be used for very efficiently isolating DNA due to its broad surface area. Thus, according to the method of isolating nucleic acid of the present invention, nucleic acid can be very efficiently isolated using a carbon nanotube without using a chaotropic material.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of isolating nucleic acid using a carbon nanotube, the method comprising:
contacting a mixture of a sample containing nucleic acid and a salt solution with a carbon nanotube to form a nucleic acid-carbon nanotube composite;
washing the nucleic acid-carbon nanotube composite with a washing buffer; and
eluting the nucleic acid from the nucleic acid-carbon nanotube composite.

2. The method of claim 1, wherein the sample containing nucleic acid is a biological material.

3. The method of claim 1, wherein salt of the salt solution is at least one selected from the group consisting of NaCl, MgCl₂, KCl, CaCl₂, and a combination thereof.

4. The method of claim 3, wherein the concentration of the salt is 0.5-5 M.

5. The method of claim 1, wherein the carbon nanotube is coated with a carboxylic group.

6. The method of claim 1, wherein the washing is performed with a washing buffer containing ethanol and EDTA.

7. The method of claim 1, wherein the nucleic acid is eluted with at least one selected from the group consisting of water, Tris-buffer, and PBS.

8. The method of claim 1, wherein the mixture of a sample containing nucleic acid and a salt solution includes 0-40% PEG.


SEQUENCE LISTING (2 sequences)

SEQ ID NO: 1 - DNA, 23 bp, Artificial Sequence
  forward primer
gctttggggcatggacattgacc

SEQ ID NO: 2 - DNA, 23 bp, Artificial Sequence
  reverse primer
agcagaggcggtgtcgaggagat